(19) United States
(12) Patent Application Publication
(10) Pub. No.: US 2010/0210735 A1
(43) Pub. Date: Aug. 19, 2010

(54) Modulating the Activity of Nuclear Receptors in Order to Treat Hypoxia-Related Disorders

(75) Inventor: Jianrong Lu, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: UNIVERSITY OF FLORIDA RESEARCH FOUNDATION, INC., Gainesville, FL (US)

(21) Appl. No.: 12/672,289

(22) PCT Filed: Aug. 6, 2008
(86) PCT No.: PCT/US2008/072312

§ 371 Date: Mar. 24, 2010

Related U.S. Application Data
(60) Provisional application No. 60/954,139, filed on Aug. 6, 2007.

(51) Int. Cl. A61K 31/05; C12Q 1/02; A61P 35/00

(52) U.S. Cl. 514/733; 435/29

(57) ABSTRACT

The subject invention provides methods for treating hypoxia-related conditions by modulating the activity of certain nuclear receptors. Specifically exemplified herein are materials and methods for treating cancer and other pathological conditions involving hypoxia and/or abnormal or excessive neovascularization.

### BACKGROUND OF INVENTION

Hypoxia (low oxygen) occurs when tumor growth outstrips new blood vessel formation. The development of intratumoral hypoxia is a common hallmark of rapidly growing solid tumors. Cancer cells undergo adaptation, including neo-vascularization, to persist in the hostile hypoxic environment. Other diseases such as diabetic retinopathy and macular degeneration also involve excessive/abnormal neo-vascularization

The adaptive response to hypoxia is mediated by the hypoxia-inducible factor (HIF) transcription factor, a master regulator of hypoxic gene expression and oxygen homeostasis. HT orchestrates transcriptional activation of a myriad of genes that facilitate crucial adaptive mechanisms, such as increased glucose uptake, a switch to glycolytic metabolism, angiogenesis, as well as invasion and metastasis.

Mounting experimental and clinical evidence suggests that HIF plays a causal role in tumor survival, growth, and progression. It has been shown that attenuation of hypoxic response leads to markedly diminished tumor growth. However, HIF is difficult to inhibit directly by drug-like small molecules. Currently, the available tools for use against HT are very limited.

A number of anticancer agents that target oncogenic signal transduction pathways can indirectly inhibit HIF through repression of protein synthesis (Harris, A. L. “Hypoxia—a key regulatory factor in tumour growth” Nat Rev Cancer 2, 38-47 (2002); Semenza, G. L. “Targeting HIF-1 for cancer therapy” Nat Rev Cancer 3, 721-732 (2003)). Recently, chetomin, a small natural product capable of binding to p300 and disrupting the HIF-p300 interaction, was shown to attenuate hypoxia-inducible gene transcription and tumor growth (Kung, A. L., Zabludoff S D, France D S, Freedman S J, Tanner E A, Vieira A, Cornell-Kennon S, Lee J, Wang B, Wang J et al. “Small molecule blockade of transcriptional coactivation of the hypoxia-inducible factor pathway” Cancer Cell, 6, 33-43 (2004)). Unfortunately, development of chetomin as a therapeutic may be limited by its toxicity. Currently very few inhibitors have been elucidated in the regulation of HIF activity.

Genetic studies demonstrate that p300/CBP are responsible for a subset of HIF-stimulated genes and suggest the existence of additional coactivating mechanisms (Kasper, L. H. et al. (2005) “Two transactivation mechanisms cooperate for the bulk of HIF-1-responsive gene expression” EMBO J24:3846-3858). Moreover, although HIF stabilization and activation under hypoxia are evident in almost all cell types, expression of many HIF target genes exhibits a cell-type specific pattern. HIF alone cannot account for this cell type specificity. Recent in vivo characterization of a hypoxia-inducible promoter suggested that the HIF-responsive element (HRE) is required but not sufficient for the hypoxic response. Additional ancillary cis elements are essential in the process, even though the corresponding trans factor(s) has not been identified (Kajimura, S. et al. (2006) “Understanding hypoxia-induced gene expression in early development: in vitro and in vivo analysis of hypoxia-inducible factor 1-regulated zebra fish insulin-like growth factor binding protein 1 gene expression” Mol Cell Biol 26:1142-1155).

HIF is heterodimeric, comprising an oxygen-regulated α-subunit (HIF1α or HIF2α) and a constitutively expressed and stable β-subunit (HIFβ) (Harris, A. L. “Hypoxia—a key regulatory factor in tumour growth” Nat Rev Cancer 2, 38-47 (2002); Semenza, G. L. “Targeting HIF-1 for cancer therapy” Nat Rev Cancer 3, 721-732 (2003); Gordan, J. D. and Simon M. C. “Hypoxia-inducible factors: central regulators of the tumor phenotype” Current Opinion in Genetics & Development, 17, 71-77 (2007)). Under normal oxygen tensions, the HIFα subunit is subjected to prolyl hydroxylation catalyzed by a set of oxygen- and ferrous ion-dependent prolyl hydroxylases. Hydroxylated HIFα is then recognized by the tumor-suppressor protein von Hippel-Lindau (VHL), a component of an E3 ubiquitin ligase complex. Subsequently, HIFα becomes poly-ubiquitinated and targeted for rapid degradation by the proteasomal system. Oxygen deprivation, or administration of iron chelators or cobaltous ion (a classic hypoxia mimetic which competes with ferrous ion) suppresses hydroxylase activity, allowing HIFα to escape the VHL-mediated destruction and to accumulate and dimerize with the constitutively present HIFβ. The binding of the HIFα-HIFβ heterodimer along with the transcriptional coactivator p300/CBP to the cognate hypoxia-responsive element (HRE) augments the expression of hypoxic genes containing such elements within their promoters or enhancers.

Many of the HIF-dependent genes are directly responsible for malignant progression including metabolic adaptation, angiogenesis and metastasis. Cancer cells commonly rely on glycolysis for their energy needs (Gatenby, R. A. and Gillies R. J. “Why do cancers have high aerobic glycolysis?” Nat Rev Cancer. 4, 891-899 (2004); Pan J. G. and Mak, T. W. “Metabolic targeting as an anticancer strategy: Dawn of a new era?” Sci. STKE, pe14 (2007)). Glycolysis breaks down glucose to generate pyruvate. In the absence of oxygen, pyruvate is reduced to the waste product lactic acid by lactate dehydrogenase (LDH). In the presence of oxygen, pyruvate is oxidized to acetyl-CoA by the pyruvate dehydrogenase (PDH) enzymatic complex. This represents an important regulatory point in cellular energy metabolism as PDH can be switched off by the pyruvate dehydrogenase kinases (PDKs). Acetyl-CoA enters the mitochondria and the tricarboxylic acid (TCA) cycle, leading to the efficient production of ATP through oxidative phosphorylation (OXPHOS).

Under hypoxia, depletion of oxygen, the substrate for OXPHOS, will force cells to shut down the mitochondrial respiratory metabolism. In order to meet tumor cells' high energy demand, HIF strongly stimulates the expression of genes that encode glucose transporters and glycolytic enzymes (including LDH) to accelerate glucose uptake and the anaerobic glycolytic ATP production. Meanwhile, HIF attenuates both the TCA cycle and OXPHOS via direct induction of PDK1 (one of the 4 PDKs) to block conversion of pyruvate to acetyl CoA (Kim J W, Tchernyshyov I, Semenza G L, Dang C V. HIF-1-mediated expression of pyruvate dehydrogenase kinase: a metabolic switch required for cellular adaptation to hypoxia. Cell Metab, 3, 177-185 (2006); Papandreou I, Cairns R A, Fontana L, Lim A L, Denko N C. HIF-1 mediates adaptation to hypoxia by actively down-regulating mitochondrial oxygen consumption. Cell Metab 3, 187-197 (2006)).

This HIF-mediated active metabolic switch allows tumor cells to survive and grow under hypoxic environment. Furthermore, HIF activates several angiogenesis-promoting factors, including the vascular endothelial growth factor (VEGF), which is a well-proven dominant growth factor in tumor angiogenesis cascade. Ultimately, neovascularization may correct the hypoxia and nutrient depletion, and consequently facilitate tumor growth and metastasis (Pugh C W, Ratcliffe P J. Regulation of angiogenesis by hypoxia: role of the HIF system. Nat Med, 9, 677-684 (2003)).

The HIF-coordinated systemic cellular adaptation contributes substantially to the malignant tumor phenotype. There is mounting clinical evidence correlating tumor hypoxia and HIF overexpression to aggressive tumor behavior, diminished therapeutic response, and increased patient mortality in a broad range of cancer types. On the other hand, experimental attenuation of HIF activity by genetic or pharmacological approaches in tumor xenografts and transgenic models leads to markedly reduced tumor growth, demonstrating a causal role of HIF (Semenza GL. Targeting HIF-1 for cancer therapy. Nat Rev Cancer 3, 721-732 (2003)). Disruption of individual HIF downstream events such as angiogenesis and glycolysis has shown promising efficacy (Ferrara N and Kerbel R S. Angiogenesis as a therapeutic target. Nature 438, 967-974 (2005); Pan J G, Mak T W. Metabolic targeting as an anticancer strategy: Dawn of a new era? Sci. STKE, pe14 (2007)).

Hormone therapy has been used for decades to induce breast cancer regression. Anti-estrogen administration (e.g. estrogen receptor (ER) antagonist tamoxifen) is applied on the rationale that estrogens stimulate proliferation of breast cancer cells. Paradoxically, the synthetic estrogen Diethylstilbestrol (DES) was used clinically to obtain cancer remissions. High-dose DES was generally considered the endocrine therapy of choice in women with advanced breast cancer prior to the introduction of tamoxifen in the 1970s. Subsequently, DES was largely replaced by tamoxifen. However, the acceptance of tamoxifen as preferable to DES was based not on a superior efficacy, but rather on its lower incidence of side effects. In fact, the efficacy of the ER antagonist tamoxifen and the ER agonist DES was comparable. Survival was even modestly better for women treated with DES (Ingle J N. Estrogen as therapy for breast cancer. Breast Cancer Res, 4, 133-136 (2002)). The rationale for the use of DES was totally empirical.

Breast cancers often progress from an early ER-positive stage to an advanced ER-negative stage. ER-negative breast cancers are resistant to anti-estrogen treatment and generally have a worse prognosis. Hypoxia can promote cancer cell dedifferentiation and downregulation of ER expression (Stoner M, Saville B, Wormke M, Dean D, Burghardt R, Safe S. Hypoxia induces proteasome-dependent degradation of estrogen receptor alpha in ZR-75 breast cancer cells. Mol Endocrinol. 16, 2231-2242 (2002); Helczynska K, Kronblad A, Jogi A, Nilsson E, Beckman S, Landberg G, et al. Hypoxia promotes a dedifferentiated phenotype in ductal breast carcinoma in situ. Cancer Res. 63, 1441-1444 (2003)). Highly hypoxic tumors are most likely to be ER-negative (Chi, J. T. et al. Gene expression programs in response to hypoxia: Cell type specificity and prognostic significance in human cancers. PLoS Med. 3, e47 (2006)). On the other hand, expression of estrogen-related receptor α (ERRα) shows an inverse correlation with expression of ERα. Increased ERRα levels appear to be associated with ER-negative tumor status. Indeed, ERRα is also a potent prognosis indicator in human breast carcinoma and is significantly associated with an increased risk of recurrence and adverse clinical outcome (Ariazi E A, Clark G M, Mertz J E. Estrogen-related receptor alpha and estrogen-related receptor gamma associate with unfavorable and favorable biomarkers, respectively, in human breast cancer. Cancer Res. 62, 6510-6518 (2002); Suzuki T, Mild Y, Moriya T, Shimada N, Ishida T, Hirakawa H, Ohuchi N, Sasano H. Estrogen-related receptor alpha in human breast carcinoma as a potent prognostic factor. Cancer Res. 64, 4670-4676 (2004)). In addition, levels of ERRα expression significantly correlate with both stage and histological grade of breast, ovarian, endometrial, prostate, and colorectal cancers (Tremblay A M, Giguere V. 2007. The NR3B subgroup: an ovERRview. Nucl Recept Signal. 5:e009).

ERR stimulates expression of genes involved in mitochondrial biogenesis and OXPHOS (Tremblay A M, Giguere V. 2007. The NR3B subgroup: an ovERRview. Nucl Recept Signal.5:e009). This enhanced mitochondrial oxidative capacity is mainly toward fatty acid oxidization, as ERR activates medium chain acetyl coenzyme A dehydrogenase (MCAD), a pivotal enzyme involved in fat oxidation, and in the meantime upregulates PDK4 to prevent glucose oxidative metabolism (Wende A R, Huss J M, Schaeffer P J, Giguere V, Kelly D P. PGC-1 alpha coactivates PDK4 gene expression via the orphan nuclear receptor ERRalpha: a mechanism for transcriptional control of muscle glucose metabolism. Mol Cell Biol. 25, 10684-10694 (2005); Araki M, Motojima K. Identification of ERRalpha as a specific partner of PGC-1alpha for the activation of PDK4 gene expression in muscle. FEBS J. 273, 1669-1680 (2006); Zhang Y, Ma K, Sadana P, Chowdhury F, Gaillard S, Wang F, McDonnell D P, Unterman T G, Elam M B, Park E A. Estrogen-related receptors stimulate pyruvate dehydrogenase kinase isoform 4 gene expression. J Biol Chem. 281, 39897-39906 (2006)). The latter is reminiscent of HIF's activation of PDK1 (Kim J W, Tchernyshyov I, Semenza G L, Dang C V. HIF-1-mediated expression of pyruvate dehydrogenase kinase: a metabolic switch required for cellular adaptation to hypoxia. Cell Metab, 3, 177-185 (2006); Papandreou I, Cairns R A, Fontana L, Lim A L, Denko N C. HIF-1 mediates adaptation to hypoxia by actively downregulating mitochondrial oxygen consumption. Cell Metab 3, 187-197 (2006)), which is crucial for the switch from glucose oxidation to glycolysis under hypoxia.

### BRIEF SUMMARY

The subject invention provides methods for treating proliferative diseases and other hypoxia-related conditions by modulating the activity of nuclear receptors. Specifically exemplified herein are materials and methods for treating cancer and other pathological conditions involving hypoxia and/or abnormal or excessive neovascularization.

In one embodiment, the materials and methods of the subject invention can be used to interfere with the ability of cells, including tumor cells, to adapt to the low oxygen environment that occurs when, for example, tumor growth outstrips new blood vessel formation. By interfering with the ability of cells to adapt to this hypoxic environment, the materials and methods of the subject invention can be used to, for example, inhibit the growth and spread of tumors.

In accordance with the subject invention, it has been found that hypoxia-inducible factor (HIF) specifically interacts with a subfamily of nuclear hormone receptors, namely, the estrogen-related receptors (ERRs). This interaction stimulates HIF-dependent gene transcription and, in accordance with the subject invention, it has been found that ERRs are required for HIF's function. Thus, in accordance with one embodiment of the subject invention, tumor growth is inhibited by administering a compound that interacts with an ERR thereby inhibiting the ability of HIF to promote expression of hypoxic genes, including those involved in tumor angiogenesis.

In a further embodiment, the subject invention provides biologically active compounds as well as compositions comprising these compounds. Advantageously, certain compositions of the subject invention are useful in, for example, inhibiting tumor growth and spread. In a specific embodiment, the compounds and compositions of the subject invention can be used in the treatment of cancer, including multi-drug resistant cancers.

The methods of the subject invention can be used in the treatment of an animal hosting cancer cells including, for example, inhibiting the growth of tumors in a mammalian host. More particularly, the methods of the present invention can be used for inhibiting in a human the growth of tumor cells, including cells of breast, colon, CNS, ovarian, renal, prostate, liver, pancreatic, uterine, or lung tumors. The mechanisms for achieving anticancer activity exhibited by the subject compounds would lead a person of ordinary skill in the art to recognize the applicability of the subject compounds, compositions, and methods to additional types of cancer as described herein.

Additional embodiments of the subject invention provide materials and methods for treating conditions wherein enhanced angiogenesis is desired. In these embodiments, such as for wound healing or treating cardiac ischemia, ERR activity is enhanced according to the subject invention. Other such disorders that may be treated in this manner according to the subject invention include, but are not limited to, rheumatoid arthritis and psoriasis.

In a further embodiment, the subject invention provides materials and methods for promoting weight loss. Specifically, inhibition of ERR can be used for treating and/or preventing obesity.

### BRIEF DESCRIPTION OF THE DRAWINGS

FIG. 1A-E—HIF-ERR interaction in vitro and i.

1A) HIF heterodimers bind to all three ERRs. In vitro translated HIF proteins were mixed with GST or GST-ERR fusions and assayed for binding.

1B) HIF-ERR interaction is resistant to DES and 4HT treatment. The binding assay was performed in the presence of DES (50 μM) or 4HT (10 μM).

1C) The DBD of ERR is responsible for HIF association. (Top) Diagram of deletion mutants of ERRβ, N, N terminus; D, DBD; L, LBD. (Middle) Heterodimeric HIF associates with ERR DBD. (Bottom) Gel showing loading of GST-ERR fusion proteins.

1D) Exogenous HIF and ERR interact in vivo. Flag-ERRs and HA-HIF1α were coexpressed in COS cells. The cellular lysates were subjected to anti-Flag immunoprecipitation. HA-HIF1α was enriched in the ERR precipitates.

1E) Endogenous ERR and HIF are present in the same complex. Cellular lysates from MDA-MB-435 cells treated with DP were subjected to immunoprecipitation with anti-ERRα or a control antibody (anti-HA). The pellets were analyzed for the presence of HIF1α by blotting with the anti HIF1α antibody.

FIG. 2A-F—ERRs enhance HIF-mediated gene transcription.

2A) ERRα is recruited to HIF-bound genes under hypoxia. ChIP assay was carried out for VEGF, PDK1, PGK1 (HIF target genes), and β-actin (negative control). Sheared chromatin from normoxic or DP-treated MDA-MB-435 cells was precipitated with polyclonal anti-ERRα or control antibody (anti-HA). Enrichment of the indicated gene promoters was analyzed by PCR amplification with primers encompassing the defined HREs.

2B) ERRs potentiate the transcriptional activity of exogenous HIFα. HEK293 cells were transfected with a HIF-dependent firefly luciferase reporter (HRE-Luc), HIFα, and ERRs. In each transfection, we included a Renilla luciferase (Rlu) reporter drive by SV40 promoter (SV40-Rlu) to serve as a normalization control. Error bars represent standard deviations.

2C) ERRs enhance endogenous HIFα activity. HEK293 cells were transfected with HRE-Luc, SV40-Rlu (for normalization) and ERRs, and followed by administration of hypoxic mimetics.

2D and 2E) ERR and endogenous HIF synergistically activate EPO and PGK1. Luciferase reporters driven by the EPO enhancer or PGK1 promoter were introduced into HEK293 cells together with ERRβ and SV40-Rlu, cells were subsequently treated with DP.

2F) DES attenuates HIF activity. Breast cancer SKBR3 cells were transfected with HRE-Luc and SV40-Rlu and subsequently treated with DP, DES, or 4HT. Lack of effects by 4HT may be due to the redundant function of ERRα and ERRβ, or its failure to dissociate a specific coactivator(s) of ERR.

FIG. 3A-F—Inhibition of ERR attenuates hypoxic response.

For Northern blot analysis, nylon membranes carrying total RNA samples were hybridized with probes made from cDNA fragments encoding for VEGF, PGK1, lactate dehydrogenase A(LDHA), Enolase 1 (Eno1), and Glucose transporter 1 (Glut1). The 18s rRNA and β-actin served as loading controls.

3A) Northern blot analysis of MDA-MB-435 breast cancer cells cultured under severe hypoxic condition in the absence or presence of DES.

3B) Northern blot analysis of SKBR3 breast cancer cells exposed to DP and DES.

3C) Northern blot analysis of melanoma WM266 cells treated with DP and DES.

3D) Western blot analysis for HIF1 α in SKBR3 cells treated with DP and DES. HIF1α was detected with anti-HIF1α antibody. The same blot was reprobed with an antitubulin antibody. Tubulin served as a loading control.

3E) Resveratrol (RES)-reduced hypoxic gene expression in A549 lung cancer cells treated with DP and RES.

3F) Compromised hypoxic gene induction in MDA-MB-435 cells expressing dnERR.

FIG. 4A-D—Growth and angiogenesis of human breast cancer xenograft are sensitive to DES treatment in vivo.

4A) DES treatment affects kinetics of tumor growth. Two groups of mice bearing established tumor xenografts of human MDA-MB-435 breast cancer cells were treated daily with DES (dissolved in sesame oil) or control (sesame oil). Tumor volume was measured weekly.

4B) DES treatment leads to reduced tumor mass. Tumor xenografts were harvested at time of necropsy (after 3 weeks of treatment) and weighed. DES-treated tumors evidently weighed much less than the control samples. Scale Bars represent average tumor weight.

4C) DES treatment diminishes tumor angiogenesis. Tumor transplants were processed and subjected to CD34 immunostaining for blood vessels (brown staining). Left, tumor section from the control group exhibits high microvessel density. Right, DES-treated tumor contains few blood vessels. Scale bars, 65 μm.

4D) Blood vessel density in tumors is reduced by DES treatment. Microvessel density was determined by counting the number of CD34 positive blood vessels from four randomly chosen visual fields from each group of tumors. The average number in the fields was taken as the mean of vessel density of the group.

### DETAILED DESCRIPTION

Physiological responses to a low oxygen environment are critical factors in the etiology of a variety of pathological conditions. For example, the development of intratumoral hypoxia is a common hallmark of rapidly growing solid tumors. Cancer cells undergo adaptation to persist in the hostile anaerobic environment. The adaptive response to hypoxia is mediated primarily by the hypoxia-inducible factor (HIF), a master regulator of hypoxic gene expression. HIF orchestrates transcriptional activation of a myriad of genes that facilitate crucial adaptive mechanisms, such as increased glucose uptake, switch to glycolytic metabolism, and angiogenesis. Thus, HIF plays a causal role in tumor survival, growth, and progression. However, targeting HIF is a difficult endeavor and known modulators of the HIF pathway are limited.

According to the subject invention it has been found that the estrogen-related receptors (ERRs), physically and functionally interact with HIF. This interaction stimulates HIF-dependent gene transcription. Furthermore, in accordance with the subject invention it has been found that inhibition of ERRs results in attenuated induction of hypoxic genes (e.g. VEGF, the key angiogenesis factor) in cells cultured under hypoxia, and decreased growth and angiogenesis of human cancer. Advantageously, the activity of ERRs can be modulated according to the subject invention with, for example, small natural and synthetic compounds.

The activity of nuclear receptor proteins is modulated by ligand binding to their ligand binding domains that results in a conformational change leading to either up or down regulation of gene expression. The ligands include many natural and synthetic small lipophilic substances that can diffuse easily past the cell membrane. Existing ERR inhibitors include, for example, DES and resveratrol. The crystal structures of ERR ligand binding domains (LBDs) have revealed how inhibitors can promote dissociation of co-activator proteins and potential recruitment of co-repressors.

When ERR is inhibited, HIF remains inactive even if stabilized. Therefore, ERR inhibition can be effective irrespective of the mode of pathway activation by upstream events.

Unless the context indicates otherwise, as used herein, reference to “inhibition” of ERR includes the use of compounds (preferably small molecules but also including, for example, antibodies or other proteins and polynucleotides) that act directly, or indirectly, on an ERR to reduce its activity as that activity relates to the ability to induce, upregulate, or enhance the hypoxic response activity of HIF. Thus, one aspect of the present invention involves the use of compounds that act directly on ERR. Other embodiments include the use of compounds that interact with co-factors or other entities that then, in turn, result in a reduction of ERR activity.

ERRs (and other nuclear receptors) function, at least in part, through the recruitment transcriptional co-activators. In the case of ERR-induced activation of HIF, these co-activators include, for example, p160, p300/CBP and PGC1. The subject invention pertains not only to direct modulation of ERRs, but also to modulation of co-activators as well as other nuclear receptors which, for example in the context of specific cell types, are involved in the cascade of cellular events that results in the regulation of HIF. The modulation of the co-activators includes interference with the functional interaction of the co-activators and a nuclear receptor and/or HIF. The subject invention provides the first teaching of the modulation of nuclear receptors and their associated co-activators to treat proliferative diseases and other hypoxia-related conditions.

In one embodiment, the subject invention pertains to combination treatments comprising: (a) an ERR inhibitor (or an inhibitor of another nuclear receptor or related co-activator), in combination with (b) one or more other therapies (such as, for example, antitumor chemotherapeutics), preferably the combined treatment being timed so that component (a) and (b) are administered to a warm-blooded animal, especially a human (especially in need of such treatment), in combination in a quantity which is jointly therapeutically effective.

The invention also relates to a product which comprises component (a) and component (b) as defined above, in the presence or absence of one or more pharmaceutically acceptable carrier materials, as a combination preparation for simultaneous or chronologically staggered administration within a period of time which is small enough for the active compounds both of component (a) and of component (b) to mutually enhance activity.

The general terms used herein preferably have the following meanings, if not defined otherwise:

A proliferative disease is typically a tumor disease (or cancer) (and/or any metastases), wherever the tumor or the metastasis are located, including, for example, a tumor selected from the group comprising breast cancer, genitourinary cancer, lung cancer, gastrointestinal cancer, epidermoid cancer, melanoma, ovarian cancer, pancreas cancer, neuroblastoma, head and neck cancer or bladder cancer, or in a broader sense renal, brain or gastric cancer. In one embodiment, the proliferative disease is refractory to treatment with (an)other chemotherapeutic(s), especially with 5-fluorouracil and/or a microtubule stabilizing agent of the taxane class, most especially TAXOL®. In one embodiment, the tumor is refractory to treatment with other chemotherapeutics due to multidrug resistance, especially refractory to a member of the taxane class of microtubule stabilizing agents.

In a broader sense of the invention, a proliferative disease may furthermore be selected from hyperproliferative conditions such as hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty.

The word “refractory” means that the respective proliferative disease, upon treatment with a chemotherapeutic other than an ERR inhibitor, shows no or only weak antiproliferative response (no or only weak inhibition of tumor growth) after the treatment with such an agent, that is, a tumor that cannot be treated at all or only with unsatisfying results with other chemotherapeutics. The present invention, is to be understood to encompass not only (a) tumor(s) where one or more chemotherapeutics have already failed during treatment of a patient, but also (a) tumor(s) that can be shown to be refractory by other means, e.g. biopsy and culture in the presence of chemotherapeutics. Where a term like “refractory against TAXOL®.” is used hereinbefore and hereinafter, this term, in addition to the finished product, is also intended to mean paclitaxel, the active substance of TAXOL®. “Refractory to hormone treatment” or “hormone refractory”, in the case of a tumor of the genitourinary tract, especially a prostate tumor, means refractory to treatment with an antiandrogen.

TAXOL® stands for the finished product that comprises paclitaxel, but, in a broader sense, is also meant to encompass paclitaxel itself or any other paclitaxel formulation with one or more carrier material(s).

Preferably, the term refractory means that with standard dose a reduction of tumor growth by less than 50% (that is a T/C % value of equal to or more than 50%) is obtained when compared with a control without chemotherapeutic, e.g. by in vivo or in vitro measurements.

Multidrug resistant tumor disease is one where resistance to one or more chemotherapeutics, including those of the taxane class, especially TAXOL®, or the anthracycline class especially ADRIAMYCIN®, is found. The basis for this resistance is the export via an energy (especially ATP)-dependent pump located on the surface of cells of the respective tumor, especially of the P-glycoprotein family, especially P-glycoprotein (P-gp) itself. For example, alterations in the drug target, changes in the intracellular metabolism that may inactivate the compound, or changes in the physiology of the cell that would facilitate by-passing or overriding of the mechanism of drug action may lead to such resistance.

By the term “other chemotherapeutic” or “standard chemotherapeutic”, there is meant any chemotherapeutic other than an ERR inhibitor such as 5-fluorouracil (especially in the case of colorectal cancer), an anti-androgen or mitoxantrone (especially in the case of prostate cancer), or an antiestrogen, like letrozole (especially in the case of breast cancer); especially, the term refers to 5-fluorouracil or to members of the taxane class of microtubule stabilizing agents, such as TAXOTERE® or TAXOL®. “Standard treatment with other chemotherapeutics”, “other chemotherapeutic treatment” or “standard chemotherapy” is referring to treatment with at least one such “other” or “standard therapeutic”.

The administration may take place orally but, in view of better and better defined bioavailability, more preferably is made parenterally, especially intravenously, e.g. by infusion or injection. Where subsequently “infusion” is used, this means preferably intravenous infusion.

By the term “proliferating cells,” especially pathologically or abnormally proliferating cells are meant, such as tumor and/or tumor metastasis cells, especially of tumors as defined herein as being preferred.

Where “comprising” is used, this can preferably be replaced by “consisting essentially of”, more preferably by “consisting of”.

In specific examples, the subject invention provides materials and methods for treating cancer and other pathological conditions involving hypoxia. More specifically, the materials and methods of the subject invention can be used to interfere with the ability of cells, including tumor cells, to adapt to the low oxygen environment that occurs when, for example, tumor growth outstrips new blood vessel formation. By interfering with the ability of cells to adapt to this hypoxic environment, the materials and methods of the subject invention can be used to, for example, inhibit the growth and spread of tumors.

As noted above, the HIF transcription factor is a critical element in the regulation of hypoxic gene expression. In accordance with the subject invention, it has been found that HIF specifically interacts with a subfamily of nuclear hormone receptors, namely, the estrogen-related receptors (ERRs). This interaction stimulates HIF-dependent gene transcription and, in fact, it has now been found that ERRs are required for HIF's function. Thus, in accordance with one embodiment of the subject invention, tumor growth is inhibited by administering a compound that interacts with an ERR thereby inhibiting the ability of HIF to promote hypoxic gene expression.

While most cancers rely on glucose as their major energy source, it has been observed that tumor cells can use alternative energy sources for energy needs. For example, fatty acid oxidation may be a dominant bioenergetic pathway in some types of prostate and breast cancer cells. Fatty acid synthase (FAS), a key enzyme in fatty acid metabolism, is up-regulated in some cancers. Drug- and radiation-resistant cancer cells use fatty acid to support mitochondrial oxygen consumption when glucose becomes limited. By inhibiting ERR the subject invention is particularly advantageous for the treatment of those types of cancer that use fatty acid metabolism to support mitochondrial oxygen consumption.

Further, one potential scenario of breast cancer progression involves intratumoral hypoxia leading to decreased ERα levels but increased ERRα expression in some tumor cells. ERR in turn enhances HIF-mediated hypoxic response and results in a more malignant phenotype. While advanced tumors may become insensitive to anti-estrogen treatment, they are responsive to inhibition of ERR (i.e. by DES) and the associated HIF-dependent adaptive response, which provides a molecular explanation for the anticancer activity of DES seen in clinical treatment of advanced breast cancers. Like DES, resveratrol is capable of blocking the HT-induced gene transcription.

The methods of the subject invention are particularly advantageous because they can be utilized for the treatment of cancers other than those known to be directly associated with estrogen or other hormones. Thus, the methods of treating cancer according to the subject invention can be used for treating cancers other than, for example, breast cancer, ovarian cancer, prostate cancer, and colorectal cancer.

Thus, in accordance with the subject invention, nuclear receptors, including ERRS, are targets for therapeutic intervention whereby their activity is modulated by natural and synthetic compounds. In a specific embodiment, the materials and methods of the subject invention can be used for treatment of human breast cancer and other hypoxia-involved diseases.

Thus, in accordance with the subject invention, small molecules enhancing or inhibiting ERR activity facilitate the manipulation of the hypoxic response for therapeutic gains. In addition to cancer, hypoxia is implicated in other pathological conditions such as diabetic retinopathy, macular degeneration, rheumatoid arthritis, cardiac ischemic, and wound healing.

In a further embodiment, the subject invention provides materials and methods for promoting weight loss. Specifically, inhibition of ERR can be used for treating and/or preventing obesity.

### Materials and Methods

### Cell Lines and Cell Cultures

All human cancer cells were maintained in the following basal media (Invitrogen) supplemented with 10% fetal bovine serum: breast cancer cell line MDA-MB-435 in DMEM/F12, melanoma WM266 cells in McCoy's 5A, breast cancer SKBr3 in DMEM. Cells were treated with the indicated chemicals (purchased from Sigma): DP (100 uM), CoCl₂ (300 uM), DES (50 uM), 4HT (10 uM). The antibiotic G418 was added in the medium (800 ug/mL) for selection of stable dnERR transfectants. Anaerobic culture was achieved using GasPak Pouch System (BD Biosciences).

### Plasmids and Antibodies

The HIF-dependent reporter HRE-Luc was generated by inserting the two copies of synthetic HRE from PGK1 into a pGL3 luciferase vector. (Promega) Epo-Luc and PGK1-Luc were created by cloning the PCR-amplified HIF-dependent enhancer and promoter regions of Epo and PGK1, respectively, Full-length EST clones encoding ERR-α, -β, -γ, HIF-1α, -2α, -1β were used for in vitro translation (Promega TNT kit), production of GST fusions by cloning into the pGEX vector, and mammalian expression by cloning into modified pcDNA3 vectors with Flag or HA epitope tags. ERR Deletion mutants were generated by PCR. dnERR was constructed by PCR amplification of ERRβ DBD and Engrailed repression domain, followed by a three-way ligation into pcDNA3. Anti-Flag, -HA, -ERRα, and -HIF1α antibodies were obtained from Sigma, Covance, Upstate, and BD Biosciences and Novus, respectively.

### Northern Blot, GST Binding, Western Blot, IP, and ChIP

All procedures followed standard molecular biology techniques. For Northern, total RNA was prepared using Trizol (Invitrogen). For GST binding and IP, we used the following binding buffer (20 mM Tris, pH7.5, 150 mM NaCl, 0.5% NP-40, and protease inhibitor cocktail (Roche)). HIFα was not stable. In later experiments, we added proteasome inhibitor MG132 (10 uM) into the buffer. ChIP was performed using an Upstate kit—according to the manufacture's instructions.

### Tumor Xenograft Experiment

MDA-MB-435 cells (5×10⁶) were resuspended in a mixture of 100 uL of PBS and Matrigel (BD Biosciences, Bedford, Mass.; 2:1 ratio), and injected subcutaneously into the fourth mammary gland fat pad of 6- to 8-week-old female severe combined immunodeficient (SCID) mice. Two weeks after injection, tumor xenografts became palpable. The mice bearing tumor xenografts were randomly divided into two groups. One group was orally fed daily with 30 ul DES (25 ug/ul in sesame oil) per mouse, and the control group received 30 ul sesame oil. Tumors were serially measured weekly with a ruler and the tumor volumes were calculated with the following formula: volume (mm³)=width²× length/2. After 3 weeks of treatment, the mice were euthanized. All animal studies were approved by the Institutional Animal Care and Use Committee (IACUC) at the University of Florida.

### Immunohistochemistry

Tumors were dissected at time of necropsy, weighed, and fixed in 4% paraformaldehyde in PBS overnight. Samples were dehydrated in 70% ethanol, paraffin embedded, and sectioned (5 mm). Deparaffinized sections were stained for CD34 to assess blood vessel density. Briefly, samples were microwaved in sodium citrate (pH 6) for 20 min. for antigen retrieval, and treated with 3% H₂O₂ at room temperature for 20 min. to quench endogenous peroxidase activity. Sections were then blocked in 1.5% rabbit serum and incubated with rat anti-mouse CD34 antibody (1:100 dilution, clone MEC14.7, BioLegend), followed by biotinylated rabbit anti-rat IgG (1:200 dilution; Vector Laboratories). Detection was done with avidin-biotin-HRP complex (Vector laboratories) and di-aminobenzidin as chromogen. Nuclei were counterstained with hematoxylin. Similarly, the interactions of HIF with ERRα and ERRβ were not affected by DES either.

All patents, patent applications, provisional applications, and publications referred to or cited herein are incorporated by reference in their entirety to the extent they are not inconsistent with the explicit teachings of this specification.

Following are examples which illustrate procedures for practicing the invention. These examples should not be construed as limiting.

### Example 1

### Identification of HIF-Interacting Transcription Factors

Experiments were conducted to identify HIF-interacting transcription factors. The HIF pathway is highly conserved in fly. From a large-scale yeast two-hybrid interaction analysis of Drosophila proteins (Giot et al, 2003). A protein interaction map of Drosophila melanogaster. Science 302:1727-1736.), the fly HIFα homolog Sima showed association with an uncharacterized protein CG7404. Based on sequence similarity, CG7404 is the homolog of mammalian ERRs.

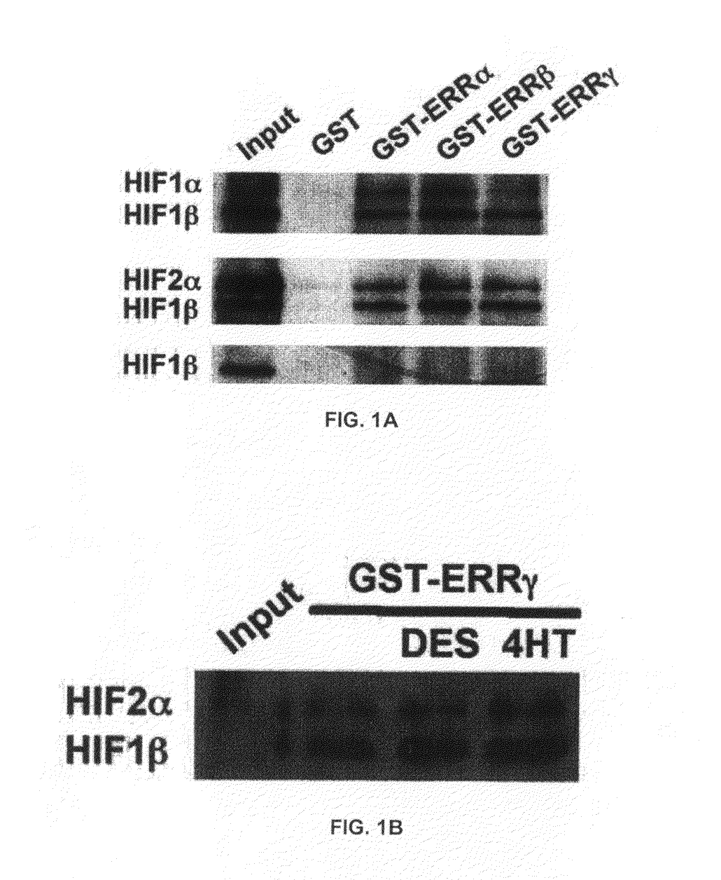

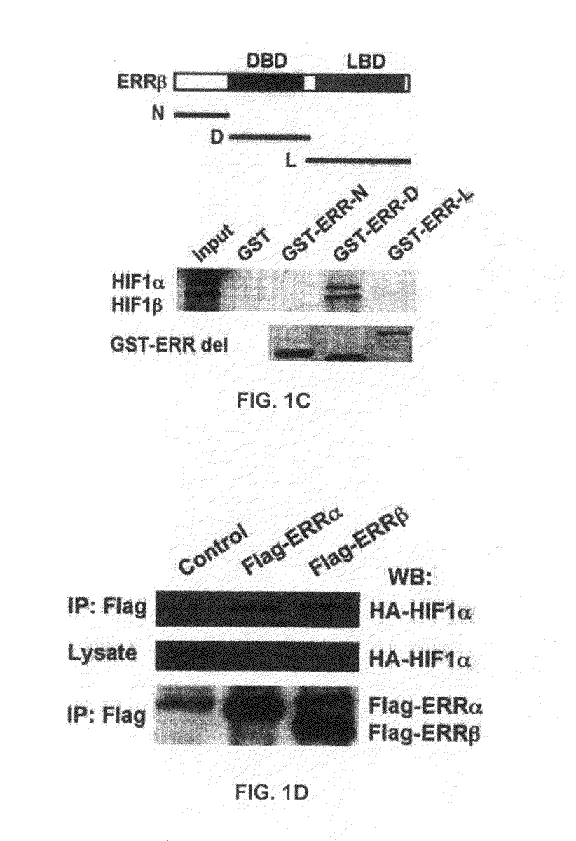

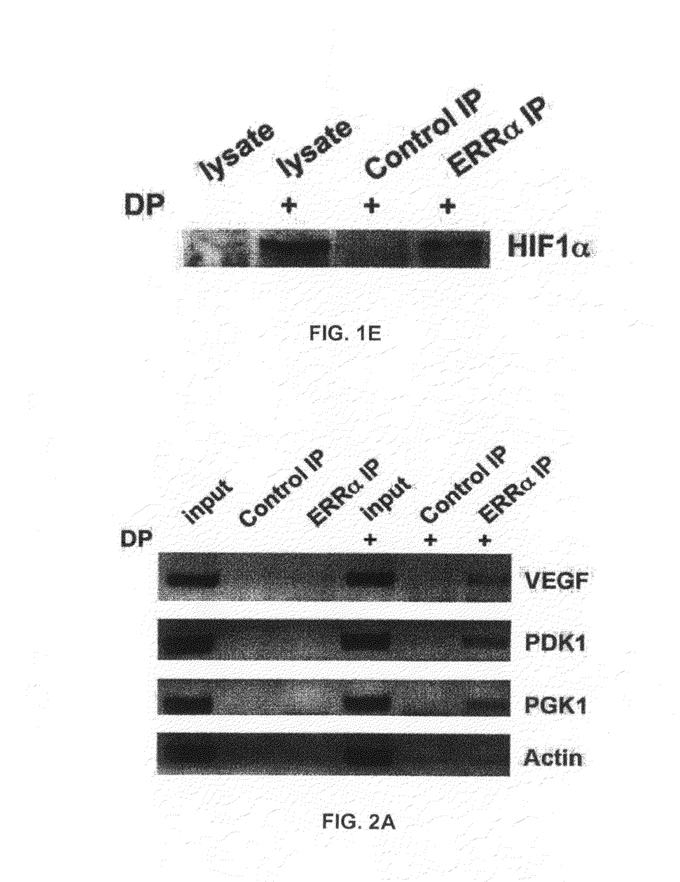

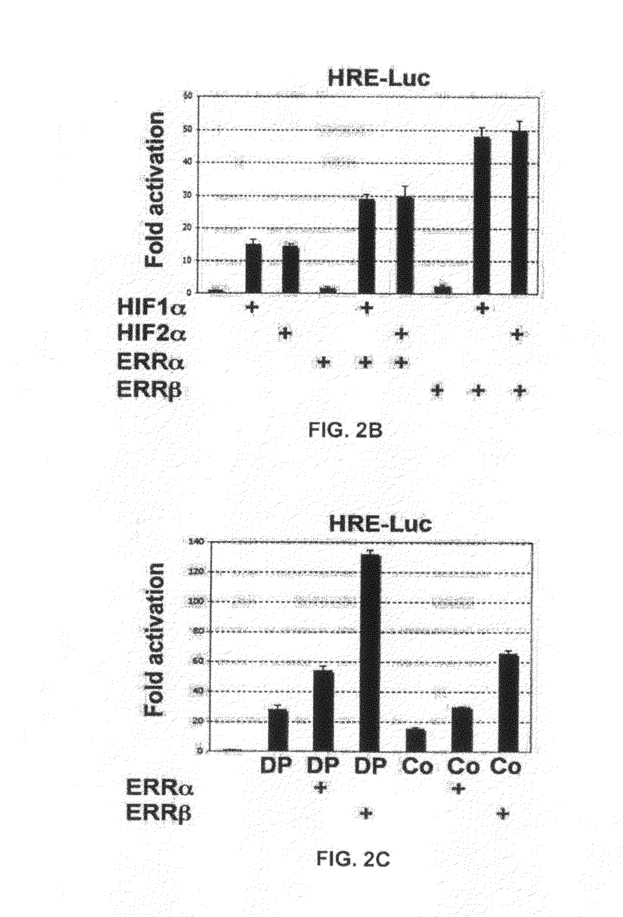

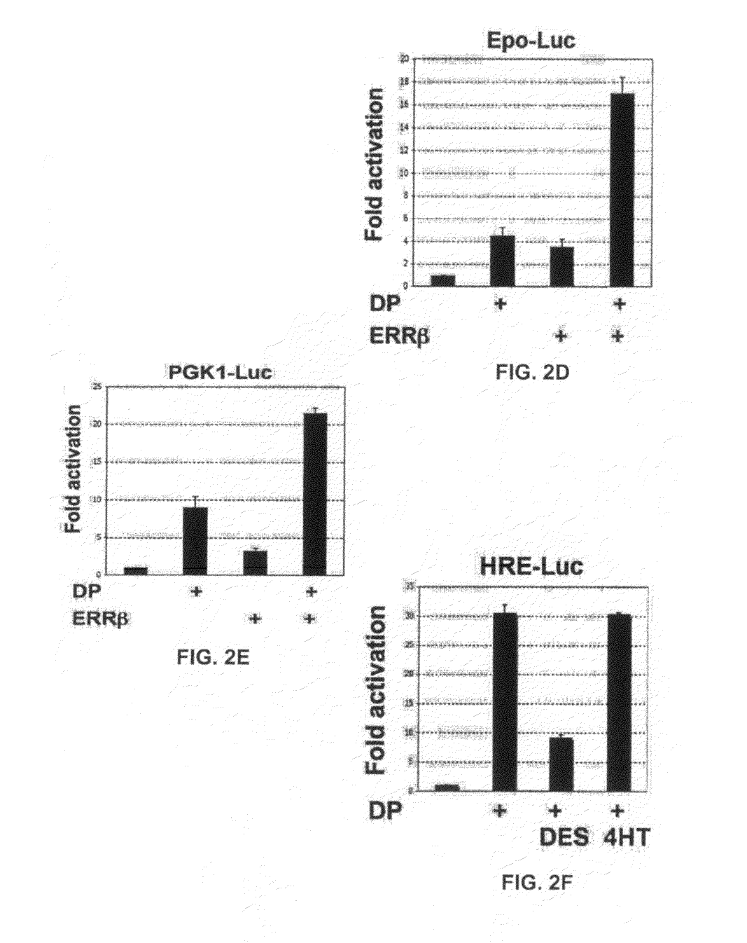

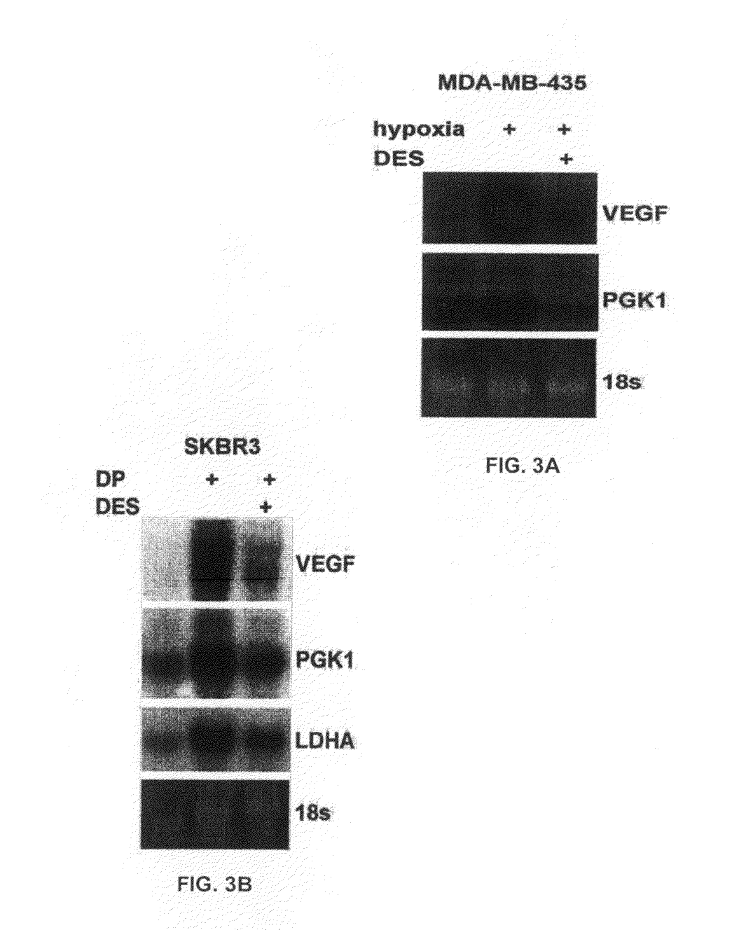

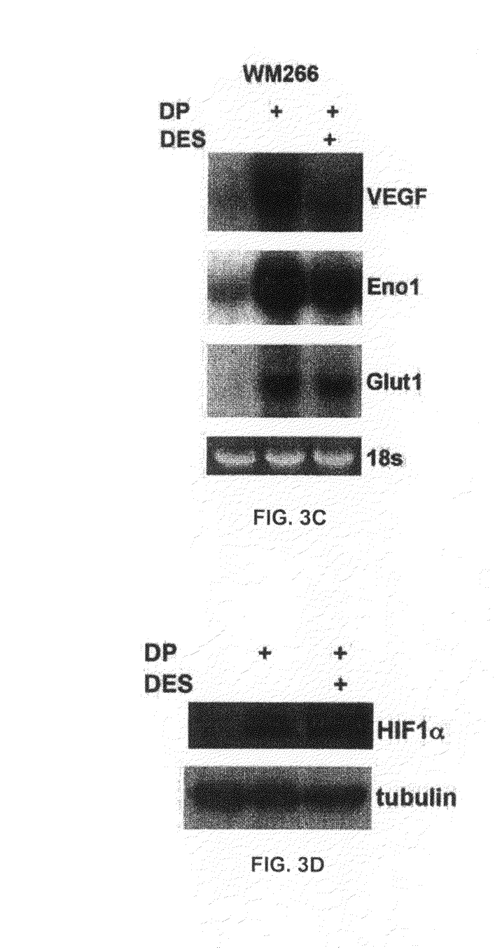

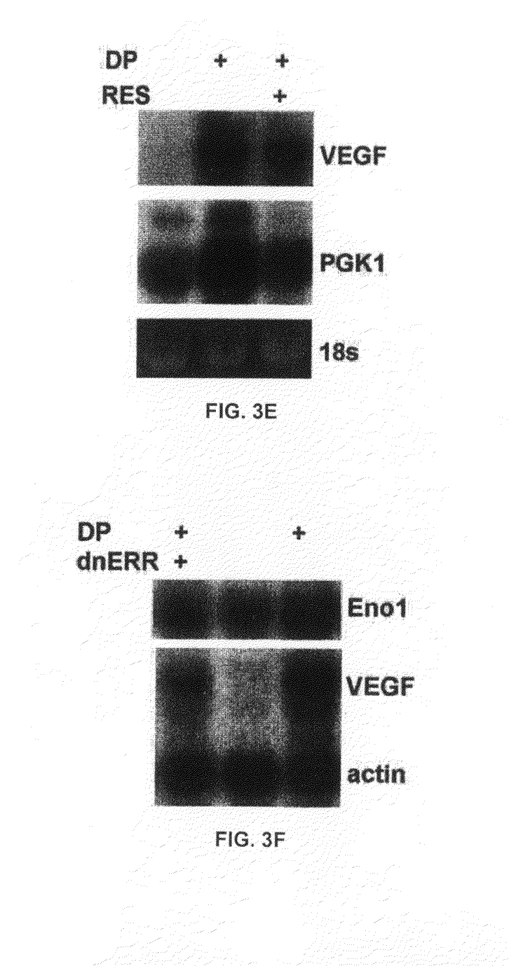

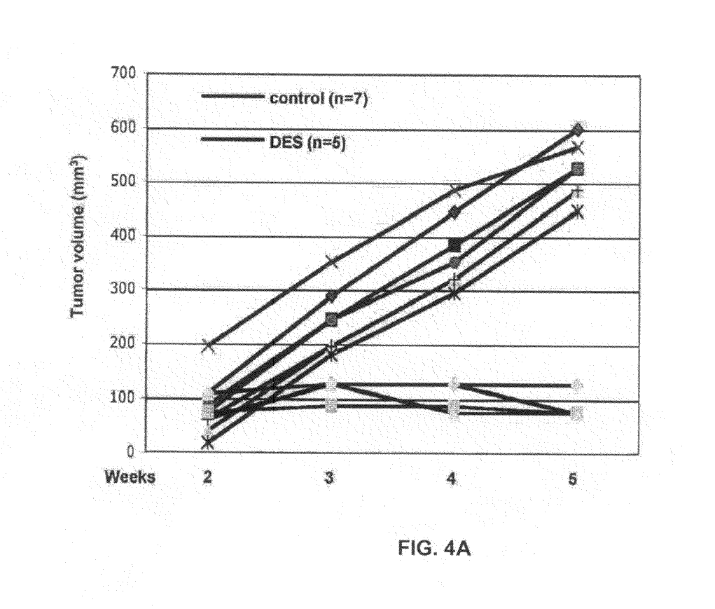

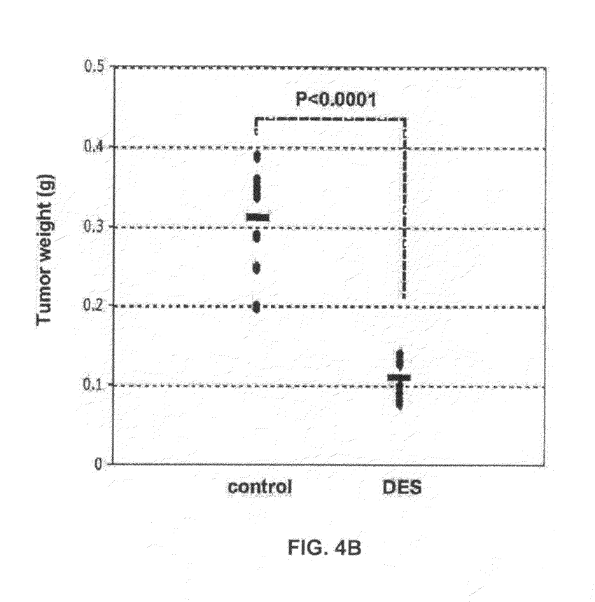

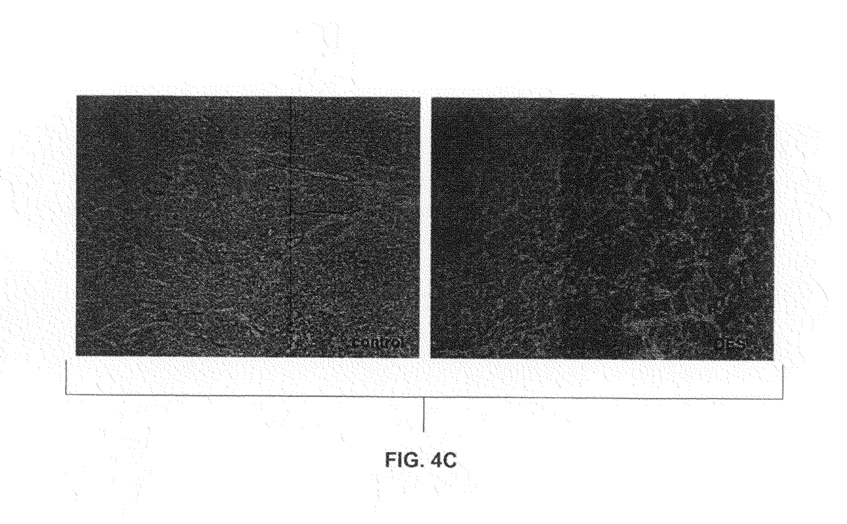

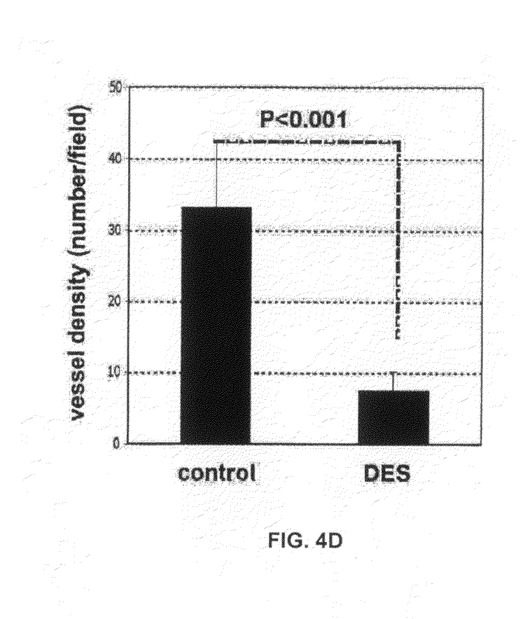

We examined whether mammalian HIF and ERR proteins interact with each other in vitro by the common glutathione S-transferase (GST) pulldown assay. We fused full-length cDNAs of ERRα, β, and γ to GST and generated fusion proteins in bacteria. We assayed them for binding to in vitro translated full-length HIF1α, HIF2α, and HIF1β. All GST-ERRs, but not GST itself, exhibited interactions with pre-mixed HIF1α-HIF1β and HIF2α-HIF1β (FIG. 1A). However, GST-ERRs did not bind to HIF1β (FIG. 1A) or HIFα when the two HIF subunits were separated, suggesting ERRs only recognized the functional HIF heterodimers.

### Example 2

### Activity of ERR Antagonists of ERRγ-HIF Association

The conformation and activity of nuclear receptors can often be modulated by small chemical molecules. The synthetic estrogen diethylstilbestrol (DES) has been found to act as an antagonist of all three ERRs by disrupting their interactions with coactivators such as p160/SRC (Tremblay et al, 2001), whereas 4-hydroxytamoxifen (4HT), a known antagonist for the estrogen receptor (ER), selectively inhibits ERRγ (Ariazi and Jordan, 2006).

These compounds were tested to determine whether they might interfere with the ERR-HT interaction. It was apparent that the ERRγ-HIF association was insensitive to the treatments with DES and 4HT (FIG. 1B), indicating that HIF may bind to a domain in ERRs which is different from the one responsible for ERRS' coactivator binding. Similarly, the interactions of HIF with ERRα and ERRβ were not affected by DES either.

### Example 3

### ERR Binding to HIF

We mapped the detailed structural requirements for ERR binding to HIF. Nuclear receptors are modular proteins that generally contain three distinct functional domains: an N-terminal domain with a ligand-independent activation function, a central zinc-finger-containing DNA binding domain (DBD) and a C-terminal ligand binding domain (LBD). We made a series of ERRβ truncation mutants, and found that the highly conserved DBD was involved in HIF binding (FIG. 1C). This result is consistent with the chemical compound experiment as DES and 4HT bind to the LBD.

### Example 4

We asked whether the ERR-HIF interaction occurred in intact cells. HIF1α and ERRs, tagged with the epitopes HA and Flag, respectively, were coexpressed in mammalian cells by transient transfection. When the ERR proteins were immunoprecipitated with the anti-Flag antibodies, HIF1α was enriched in the pellets as revealed by Western blot analysis with the anti-HA antibodies (FIG. 1D). This observation supports that the HIF and ERR proteins form complexes in vivo.

### Example 5

We further investigated whether endogenous HIF and ERR proteins might exist in the same complex. The human breast cancer MDA-MB-435 cells were treated with the iron chelator dipyridyl (DP) to stabilize the endogenous HIFα, and the cellular lysates were subjected to immunoprecipitation with anti-ERRα to enrich ERR-associated proteins. HIF1α was readily detected in the immunoprecipitates by anti-HIF1α Western blot analysis (FIG. 1E). Together, these results suggest that HIF and ERR proteins physically associate with each other both in vitro and in vivo.

### Example 6

### Recruitment of ERR Proteins in Response to Hypoxia

HIF exerts its function by binding to the HREs present in the promoter/enhancer of many hypoxic genes (Wenger et al, 2005). Given the physical association between HIF and ERR, we investigated whether the ERR proteins are present at the HIF-bound genes. We chose a few established HIF target genes with well defined HREs, such as VEGF, PDK1, and phosphoglycerate kinase 1 (PGK1), and analyzed potential in vivo promoter binding of HT and ERR by the ChIP assay. In normoxic cells, chromatin precipitated with the anti-HIF1α and -ERRα antibodies showed no enrichment of the HIF-dependent genes (FIG. 2A). However, in DP-treated cells in which HIFα was stabilized, occupancy of both HIF1α and ERRα at the HIF target gene promoters was detected (FIG. 2A), suggesting that in response to hypoxia, the ERR proteins are recruited to HIF-regulated genes.

### Example 7

### ERR proteins Augment Hypoxic Gene Expression

The presence of ERR at HT target genes raised the possibility that ERR might directly modulate HIF-mediated gene transcription. We tested this idea by reporter-based assays. A HIF-dependent luciferase reporter containing multiple HREs was readily activated by transfection of the HIF1α or -2α expression vectors (FIG. 2B). Although ERRα or -β alone did not notably affect the reporter, they strongly enhanced the transcriptional activity of HIFα (FIG. 2B). The reporter also responded robustly to increased endogenous HIF activity in cells treated with hypoxic mimetic DP or cobalt (FIG. 2C). Expression of ERRα or -β further potentiated reporter activation in these HIF-stabilized cells (FIG. 2C). Synergistic effects on the hypoxic response by ERR were also observed by using reporters carrying a natural hypoxic-responsive enhancer [e.g., erythropoietin (EPO)] or promoter (e.g., PGK1) (FIGS. 2D and E). Therefore, the ERR proteins apparently serve as a positive cofactor of HIF. Consistent with the lack of a strong interaction between ER and HIF, cotransfection of ERα (in the presence or absence of its ligand E2) had no effect on HIF's transcriptional activity.

### Example 8

### Use of ERR Inhibitors to Inhibit HIF Activity

Although ERRs are capable of strengthening HIF activity, a critical question is whether they are required for the hypoxic regulation. Genetic studies in mice have shown that mouse mutants deficient for HIFα, HIFβ, or ERRβ exhibit strikingly similar phenotypes (Luo J, et al. (1997) Placental abnormalities in mouse embryos lacking the orphan nuclear receptor ERR-beta. Nature 388:778-782; Adelman D M, Gertsenstein M, Nagy A, Simon M C, Maltepe E (2000) Placental cell fates are regulated in vivo by HIF-mediated hypoxia responses. Genes Dev 14:3191-3203; Cowden Dahl K D, et al. (2005) Hypoxia-inducible factors 1 alpha and 2alpha regulate trophoblast differentiation. Mol Cell Biol 25:10479-10491), suggesting that HIF and ERR may function in the same pathway, and ERR may be an essential component.

ERRs are constitutively active orphan receptors by interacting with a number of coactivators (e.g. SRC, PGC1, p300/CBP, and SWI/SNF chromatin remodeling complex) in the absence of ligands (Ariazi and Jordan, 2006; Perissi and Rosenfeld, 2005). It is likely that through association with HIF, ERRs bring these coactivator complexes to the proximity of HIF and hence boost HIF activity.

Because the association of ERR with its coactivators can be modified by small chemical inhibitors, we investigated whether inhibition of ERR could serve as a point of intervention in the HIF-mediated hypoxia-responsive pathway.

Indeed, activation of the HIF-dependent reporter by DP-induced HIF stabilization was severely impaired by the ERR inhibitor DES (FIG. 2F), indicating that ERRs may be required for HIF's normal function.

### Example 9

### Effect of DES on Hypoxic Response

The HIF-mediated hypoxic response is defined by transcriptional activation of many hypoxic genes. We asked whether inhibition of ERR was capable of blocking hypoxic-induced transcription of endogenous genes. As expected, when breast cancer cells (MDA-MB-435 and SKBR3) were cultured under severe hypoxia or treated with DP, known direct target genes of HIF, such as the key angiogenic factor VEGF and glycolytic enzymes, were robustly up-regulated (FIGS. 3A and B). Interestingly, in the presence of DES, this transcriptional induction was largely abolished (FIGS. 3A and B). A similar phenomenon was observed in melanoma cells (FIG. 3C).

### Example 10

### Attenuation of Transcriptional Activity of HIF Through Inhibition of ERR

We examined whether the effect caused by DES was due to its possible influence on HIFα protein stability. It seemed that under DES treatment, HIF1α was stabilized normally by DP (FIG. 3D) but remained transcriptionally incompetent. This unique effect of DES distinguished it from several HIF pathway inhibitors, which mainly decrease HIF protein levels. We conclude that inhibition of ERRs by DES attenuates the transcriptional activity of HIF.

We explored whether other ERR inhibitors might interfere with the hypoxic response as well. An interesting candidate is resveratrol, which is known to exert a wide range of activities from extending life span to suppressing tumor growth and neovascularization (Baur J A, Sinclair D A (2006) Therapeutic potential of resveratrol: the in vivo evident. Nat Rev Drug Discov 5:493-506). Resveratrol is capable of dissociating coactivator complexes from all three ERRs, albeit at higher concentrations than DES (Tremblay G B et al. (2001) Diethylstilbestrol regulates trophoblast stem cell differentiation as a ligand of orphan nuclear receptor ERR beta. Genes Dev 15:833-838). Indeed, we found that resveratrol manifested inhibitory activities on the hypoxia-induced gene transcription (FIG. 3E). This finding also raises the possibility that some of the resveratrol's effects might be attributable to its ability of inhibiting ERRs and the hypoxic response.

### Example 11

### Mechanism for Blockade of Hypoxic Response

To further attest that inhibition of ERRs was accountable for the observed effect of DES on the hypoxic response, we adopted a molecular approach to inactivate ERR. We designed a dominant negative form of ERR (dnERR) to selectively antagonize the activities of all three ERRs. We fused the DBD of ERRβ, which is sufficient for HIF binding (FIG. 1C), to a transcriptional repression domain of the Drosophila Engrailed protein (Tolkunova E N, Fujioka M, Kobayashi M, Deka D, Jaynes J B (1998) Two distinct types of repression domain in engrailed: one interacts with the groucho corepressor and is preferentially active on integrated target genes. Mol Cell Biol 18:2804-2814). The Engrailed domain possesses potent repressive activity when fused to heterologous proteins and has frequently been used to create artificial transcriptional repressors. The ERR-engrailed chimeric protein was intended to suppress HIF-mediated transcription. We expressed dnERR in the MDA-MB-435 cells by stable transfection. Although the activity of endogenous ERRs was down-regulated, the dnERR-expressing cells did not display obvious defects in gross morphology and growth. Under the hypoxic stimulus, however, induction of hypoxic genes was greatly compromised in the dnERR-expressing cells when compared with the control parental cells (FIG. 3F). Together, this finding supports that blockade of the hypoxic response pathway by pharmacological ERR inhibitors Is likely a result of specific inhibition of ERRs.

### Example 12

### Effect of ERR Inhibition on Human Tumor Cells in a Mouse Xenograft Model

The HIF-mediated hypoxic response has been shown to be critical for tumor survival and growth in vivo. To extend our studies in cultured cells, we, evaluated the effect of ERR inhibition on human tumor cells in a mouse xenograft model.

Human breast cancer MDA-MB-435 cells were implanted subcutaneously into immunodeficient mice and allowed to grow for two weeks to establish xenografts prior to initiation of DES treatment. Each mouse was fed with either vehicle (oil) or 750 μg DES daily. A slightly lower dose of DES (500 μg/mouse) has previously been shown to cause placental defects in pregnant female mice (Tremblay et al, 2001. Diethylstilbestrol regulates trophoblast stem cell differentiation as a ligand of orphan nuclear receptor ERR beta. Genes Dev 15:833-838), which mimic the ERRβ knockout mutants (Luo et al, 1997. Placental abnormalities in mouse embryos lacking the orphan nuclear receptor ERR-beta. Nature 388:778-782), strongly suggesting that this dosage is sufficient for inactivation of ERRs in vivo. There is a dramatic reduction in tumor volumes and growth rate in DES-administrated mice when compared to the vehicle-treated control animals (FIG. 4A). In fact, the tumor xenografts essentially stopped growing upon DES treatment. At the end of treatment, tumors were excised and analyzed. In comparison to tumors in vehicle-treated mice, DES-treated xenografts were much smaller and about 3 times lighter (FIG. 4B).

### Example 13

### Reducing Angiogenesis by Inhibiting ERR

As angiogenesis is a key part of hypoxic response and is required to support tumor growth beyond a certain threshold size, we assessed tumor vascularization by performing immunohistochemical staining using anti-CD34 antibody.

Mouse CD34 is present on endothelial cells and hematopoietic progenitor cells. The antibody recognizes endothelium in vivo, particularly on small vessels and newly formed capillaries and developing vascular structures.

CD34 staining revealed abundant vascular capillary formation within the control tumors (FIG. 4C), and a clear reduction of micro vascular density in tumors from the DES-treated animals (FIG. 4D).

Therefore, inhibition of ERR results in retarded tumor growth and diminished tumor angiogenesis in vivo.

### Example 14

In one embodiment, the subject invention pertains to the identification of additional compounds that modulate the activity of estrogen-related receptors (or other nuclear receptor or co-activators) and the use of these compounds to treat proliferative diseases and/or other hypoxia-related conditions.

Thus, one skilled in the art having the benefit of the instant disclosure can utilize standard procedures to identify compounds that can be used in the methods of the subject invention. For example, one typical procedure involves the in vitro protein-protein interaction assay. Thus, for example, the interaction is established using GST-ERRs to bind peptides from the co-activators; or using GST-co-activators to bind ERR peptides. Such interaction is then subjected to high-throughput screens to identify compounds that can dissociate the complex. This method has been widely used for identifying new agonists or antagonists for nuclear receptors.

In a preferred embodiment, inhibitors of nuclear receptors (or functionally related molecules) identified as described above are then subjected to further assays to confirm activity for treating cancer and/or other hypoxia related conditions.

The compounds thus identified can then be used in the methods and pharmaceutical compositions of the subject invention.

### Example 15

### Formulation and Administration

Therapeutic application of the compositions can be accomplished by any suitable therapeutic method and technique presently or prospectively known to those skilled in the art.

The dosage administration to a host in the above indications will be dependent upon the identity of the cancer cells, the type of host involved, its age, weight, health, kind of concurrent treatment, if any, frequency of treatment, and therapeutic ratio.

The compounds of the subject invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the bioactive compound(s) is combined with a suitable carrier in order to facilitate effective administration of the composition. Examples of such carriers for use in the invention include ethanol, dimethyl sulfoxide, glycerol, silica, alumina, starch, and equivalent carriers and diluents.

The present invention also relates to the manufacture of a pharmaceutical formulation. The ERR inhibitor may be used, for example, for the preparation of pharmaceutical compositions that comprise an effective amount of the active ingredient together or in admixture with a significant amount of inorganic or organic, solid or liquid, pharmaceutically acceptable carriers.

The pharmaceutical compositions according to the invention are those for enteral, such as nasal, rectal or oral, or preferably parenteral, such as intramuscular or intravenous, administration to a warm-blooded animal (human or animal), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration; preferably, the dose is one of the preferred doses as defined above, being accommodated appropriately where pediatric treatment is intended.

The pharmaceutical compositions comprise from about 0.00002 to about 95%, especially (e.g. in the case of infusion dilutions that are ready for use) of 0.0001 to 0.02%, or (for example in case of infusion concentrates) from about 0.1% to about 95%, preferably from about 20% to about 90%, active ingredient (weight by weight, in each case).

Pharmaceutical compositions according to the invention may be, for example, in unit dose form, such as in the form of ampoules, vials, suppositories, dragees, tablets or capsules.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are preferably used, it being possible, for example in the case of lyophilized compositions that comprise the active ingredient alone or together with a pharmaceutically acceptable carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8 to 22, especially from 12 to 22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, .beta.-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefosse, Paris), “Miglyol 812” (triglyceride of saturated fatty acids with a chain length of C.sub.8 to C.sub.12, Huls A G, Germany), but especially vegetable oils, such as cottonseed oil, almond oil, olive oil, castor oil, sesame oil, soybean oil and more especially groundnut oil.

The injection or infusion compositions are prepared in customary manner under sterile conditions; the same applies also to introducing the compositions into ampoules or vials and sealing the containers.

The pharmaceutically acceptable organic solvent used in a formulation according to the invention may be chosen from any such organic solvent known in the art. Preferably the solvent is selected from alcohol, e.g. absolute ethanol or ethanol/water mixtures, more preferably 70% ethanol, polyethylene glycol 300, polyethylene glycol 400, polypropylene glycol or N-methylpyrrolidone, most preferably polypropylene glycol or 70% ethanol or especially polyethylene glycol 300.

Formulations must be diluted in an aqueous medium suitable for intravenous administration before being administered to a patient.

The infusion solution preferably must have the same or essentially the same osmotic pressure as body fluid. Accordingly, the aqueous medium preferably contains an isotonic agent which has the effect of rendering the osmotic pressure of the infusion solution the same or essentially the same as body fluid.

The isotonic agent may be selected from any of those known in the art, e.g. mannitol, dextrose, glucose and sodium chloride. Preferably the isotonic agent is glucose or sodium chloride. The isotonic agents may be used in amounts which impart to the infusion solution the same or essentially the same osmotic pressure as body fluid. The precise quantities needed can be determined by routine experimentation and will depend upon the composition of the infusion solution and the nature of the isotonic agent. Selection of a particular isotonic agent is made having regard to the properties of the active agent.

Infusion solutions may contain other excipients commonly employed in formulations to be administered intravenously. Excipients include antioxidants.

Infusion solutions may be prepared by mixing an ampoule or vial of the formulation with the aqueous medium, e.g. a 5% w/v glucose solution in WFI or especially 0.9% sodium chloride solution in a suitable container, e.g. an infusion bag or bottle.

The infusion solution, once formed, is preferably used immediately or within a short time of being formed, e.g. within 6 hours.

Containers for holding the infusion solutions may be chosen from any conventional container which is non-reactive with the infusion solution. Glass containers made from those glass types afore-mentioned are suitable although it may be preferred to use plastics containers, e.g. plastics infusion bags.

Plastics containers may be principally those composed of thermoplastic polymers. Plastics materials may additionally comprise additives, e.g. plasticisers, fillers, antioxidants, antistatics and other additives conventional in the art.

A wide range of container sizes may be employed. It is preferred to use containers which can accommodate between about 250 to 1000 ml of infusion solution, but preferably about 50 to about 120 ml.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

Suitable pharmaceutically acceptable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, also carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragee cores are provided with suitable, optionally enteric, coatings, there being used, inter alia, concentrated sugar solutions which may comprise gum arabic, talc, polyvinylpyrrolidone, polyethylene glycol and/or titanium. dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticiser, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, and also stabilizers and/or antibacterial agents may be added. Dyes or pigments may be added to the tablets or dragee coatings or the capsule casings, for example for identification purposes or to indicate different doses of active ingredient. To provide for the administration of such dosages for the desired therapeutic treatment, pharmaceutical compositions of the invention will advantageously comprise between about 0.1% and 45%, and especially, 1 and 15%, by weight of the total of one or more of the active compounds based on the weight of the total composition including carrier or diluent. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

I claim:

1. A method for treating a hypoxia-related condition wherein said method comprises administering, to an individual in need of such treatment, a compound that modulates either the activity of a nuclear receptor or the activity of a co-activator having a functional interaction with a nuclear receptor, such that the activity of hypoxia-inducible factor (HIF) is either upregulated or down-regulated.

2. The method, according to claim 1, which results in the downregulation of HIF.

3. The method, according to claim 2, which is used to treat a proliferative disease.

4. The method, according to claim 3, wherein said proliferative disease is cancer.

5. The method, according to claim 4, wherein said cancer is a multidrug resistant cancer.

6. The method, according to claim 3, used to inhibit the growth of a tumor.

7. The method, according to claim 1, wherein said nuclear receptor is an estrogen-related receptor.

8. The method, according to claim 7, wherein said estrogen-related receptor is ERRα.

9. The method, according to claim 7, which comprises administering an inhibitor of the estrogen-related receptor.

10. The method, according to claim 9, which comprises administering DES and/or resveratrol.

11. The method, according to claim 1, which is used to treat a non-hormone related cancer.

12. The method, according to claim 1, which is used to treat a hypoxia-related condition that is not cancer.

13. The method, according to claim 1, which is used to treat a condition by upregulating HIF.

14. A method for identifying a compound that can be used to treat a hypoxia-related condition wherein said method comprises:
a) conducting an assay to identify compounds that inhibit either a nuclear receptor or a co-activator of a nuclear receptor, and
b) conducting one or more assays with an inhibitor as identified in step a) to confirm the ability of the inhibitor to downregulate HIF.

15. A method for treating a hypoxia-related condition wherein said method comprises administering to an individual in need of such treatment a compound that was identified by the method of claim 14.

16. A pharmaceutical composition that comprises a compound identified by the method of claim 14, wherein said composition further comprises a pharmaceutically acceptable carrier.
